SEQUENCE LISTING (20 sequences)

SEQ ID NO: 1 - protein, 244 aa, Plasmodium yoelii
MDEEQRHIKLSKKHATPVVLVVGDPGRVDKIKVLCDSYVDLACNREYKSVECHYKGQKFLCVSHGVGSAGCAICFEELINIGAKVIIRAGSCGSLQPESIKRGDLCVCNAAVREDRVTHMMIHSDFPAVADYEVYSTLLKCAEELNVKVHNGISLSSDLYYPHSIIPTRLLDYSKANVAVVEMELSTLMVMGTLKKVKTGGIFIVDGCPLKWDEGDFDNVLAADRLENMIKISLEACARLSEKY

SEQ ID NO: 2 - protein, 244 aa, Plasmodium berghei
MDEEQRHIKLSKKHATPVVLVVGDPGRVDKIKVLCDSYVDLAYNREYKSVECHYKGQKFLCVSHGVGSSGCAICFEELINIGAKVIIRAGSCGSLQPESIKRGDLCVCNAAVREDRVTHMMIHSDFPAVADYEVYSTLLKCAEELNVKVHNGISLSSDLYYPHSIIPTRLLDYSKANVAVVEMELSTLMVMGTLKKVKTGGIFIVDGCPLKWDEGDFDNVLAADRLENMIKISLEACAKLSKKY

SEQ ID NO: 3 - protein, 245 aa, Plasmodium vivax
MEGEMQRHIKLTKAQTTPVVLVVGDPGRVDKVKVLCDSYVDLAYNREYKSVECTYKGQKFLCVSHGVGSAGCAICFEELMNNGAKVIIRAGSCGSLQPTQMKRGDICICNAAVREDRVSHLMIYSDFPAVADYEVYATLNQVAEELKVPVFNGISLSSDMYYPHKIIPTRLEDYSKANVAVVEMEVATLMVMGTLRKVKTGGIFIVDGCPLKWDEGDFDNNLVPERLENMIKISLETCARLAKKY

SEQ ID NO: 4 - protein, 245 aa, Plasmodium knowlesi
MEEEMQRHIKLTPSQTTPVVLVVGDPGRVDKVKMLCDSYVDLAYNREYKSVECTYKGQKFLCVSHGVGSAGCAICFEELMNNGAKVIIRAGSCGSLQPTQMKRGDICICNAAVREDRVSHLMIYSDFPAVADFEVYDTLNKVAQELEVPVFNGISLSSDLYYPHKIIPTRLEDYSKANVAVVEMEVATLMVMGTLRKVKTGGIFIVDGCPLKWDEGDFDNNLVPEKLENMIKISLETCARLAKKY

SEQ ID NO: 5 - protein, 245 aa, Plasmodium falciparum
MDNLLRHLKISKEQITPVVLVVGDPGRVDKIKVVCDSYVDLAYNREYKSVECHYKGQKFLCVSHGVGSAGCAVCFEELCQNGAKVIIRAGSCGSLQPDLIKRGDICICNAAVREDRVSHLLIHGDFPAVGDFDVYDTLNKCAQELNVPVFNGISVSSDMYYPNKIIPSRLEDYSKANAAVVEMELATLMVIGTLRKVKTGGILIVDGCPFKWDEGDFDNNLVPHQLENMIKIALGACAKLATKYA

SEQ ID NO: 6 - protein, 247 aa, Plasmodium chabaudi
MSGTQRHIKLSSKNATPVALVVGDPGRVDKIKLMCDSYIELAHNREYKSIECTYKGAKFLCVSHGVGSAGAAICFEELMNIGVKAIIRAGSCGSLQPGYIKRGDLCICNAAVREDRVTHLMIYPDYPAVGDFEVYDVLLKCAEELEVKAHTGISLSSDLYYPKDILPSRLEDYSKANVAVVEMELATLMVMGGLSGVKTGGIFIVDGCPLKWKEGDFDEVLDPKKLEEMIRVSMLACVTLTNRYTPN

SEQ ID NO: 7 - protein, 239 aa, Escherichia coli
MATPHINAEMGDFADVVLMPGDPLRAKYIAETFLEDAREVNNVRGMLGFTGTYKGRKISVMGHGMGIPSCSIYTKELITDFGVKKIIRVGSCGAVLPHVKLRDVVIGMGACTDSKVNRIRFKDHDFAAIADFDMVRNAVDAAKALGIDARVGNLFSADLFYSPDGEMFDVMEKYGILGVEMEAAGIYGVAAEFGAKALTICTVSDHIRTHEQTTAAERQTTFNDMIKIALESVLLGDKE

SEQ ID NO: 8 - DNA, 31 bp, Artificial
  primer tPyPNP-F
ggcggatccccaagaaacatgcaacccctgt

SEQ ID NO: 9 - DNA, 31 bp, Artificial
  primer tPyPNP-R
ggcgcggcgcaatgggatgagggagattttg

SEQ ID NO: 10 - DNA, 30 bp, Artificial
  primer PyPNP-WT-R
ggcgcggcgctttttattcatgcccgcttt

SEQ ID NO: 11 - DNA, 23 bp, Artificial
  primer PyPNP-5′-F
gaatatttattcaatgtttcaag

SEQ ID NO: 12 - DNA, 47 bp, Artificial
  primer PyPNP-5′-R
gctgggctgcagaggcctgttaacctacgcaaaaaggaagaatatgc

SEQ ID NO: 13 - DNA, 56 bp, Artificial
  primer PyPNP-3′-F
cgatgggtaccctcgaggctagcgagacataaataaggaacaaattcatacattac

SEQ ID NO: 14 - DNA, 24 bp, Artificial
  primer PyPNP-3′-R
gtattttgctatggatgtatacaa

SEQ ID NO: 15 - DNA, 49 bp, Artificial
  primer PyPNP-gfp-3′-R
cataaaataatgattgggcgagctcgctacgcaaaaaggaagaatatgc

SEQ ID NO: 16 - DNA, 55 bp, Artificial
  primer PyPNP-gfp-3′-F
gaacatatttattaaactgcagccgacataaataaggaacaaattcatacattac

SEQ ID NO: 17 - DNA, 26 bp, Artificial
  primer PyCSP-5′-F
aaaatggggctatttgtacataaaag

SEQ ID NO: 18 - DNA, 59 bp, Artificial
  primer PyCSP-5′-R
gctgggctgcagaggcctgttaactttaaatatgtgtgtgtatatataagttttgtttt

SEQ ID NO: 19 - DNA, 55 bp, Artificial
  primer PyCSP-3′-F
cgatgggtaccctcgaggctagcgaataaacattacacattattataaatattta

SEQ ID NO: 20 - DNA, 36 bp, Artificial
  primer PyCSP-3′-R
tcagtttagtaacttttatatttattttaaatattt

(19) United States
(12) Patent Application Publication
(10) Pub. No.: US 2010/0330126 A1
(43) Pub. Date: Dec. 30, 2010

(54) Attenuated malaria blood-stage vaccine

(76) Inventors: Kami Kim, New York, NY (US); Li-Min Ting, West New York, NJ (US)

Correspondence Address: AMSTER, ROTHSTEIN & EBENSTEIN LLP, 90 PARK AVENUE, NEW YORK, NY, 10016, US

(21) Appl. No.: 12/660,484

(22) Filed: Feb. 25, 2010

Related U.S. Application Data
(60) Provisional application No. 61/209,209, filed on Mar. 4, 2009.

(51) Int. Cl. A61K 39/015; A61P 33/06; C12N 1/10; C12N 15/87

(52) U.S. Cl. 424/272.1; 435/258.2; 435/471

(57) ABSTRACT

The present invention relates to an attenuated Plasmodium parasite having a genome comprising a non-reversible knockout of the PyPNP gene, a vaccine derived therefrom, and related methods and uses.

### CROSS-REFERENCE TO RELATED APPLICATION

This application claims the benefit of U.S. Provisional Patent Application No. 61/209,209, filed Mar. 4, 2009, the content of which is incorporated by reference.

### STATEMENT OF GOVERNMENT SUPPORT

This invention was made with government support under Grant Number R21AI052469 awarded by the National Institutes of Health and U.S. Army Research Grant Number W81XWH-05-2-0025 awarded by the Department of Defense. The government has certain rights in this invention.

### FIELD OF THE INVENTION

The present invention generally relates to an attenuated Plasmodium parasite having a genome comprising a non-reversible knockout of the PyPNP gene, a vaccine derived therefrom, and related methods and uses.

### BACKGROUND OF THE INVENTION

Throughout this application various publications are referred to by Arabic numerals in superscript. Full citations for these references may be found at the end of the specification immediately preceding the claims. The disclosures of these publications are hereby incorporated by reference in their entireties into the subject application to more fully describe the art to which the subject application pertains.

Malaria is caused by a protozoan parasite of the genus Plasmodium and is the most deadly vector-borne disease in the world. The World Health Organization estimates that there are 300-500 million clinical cases annually, resulting in approximately 1.5-2.7 million deaths⁵. Because there is no effective vaccine and drug-resistant parasites are widespread, development of alternative chemotherapeutic targets and new vaccine strategies is crucial.

Unlike their mammalian hosts, malaria parasites cannot synthesize purines de novo and depend exclusively on purine salvage and recycling for RNA and DNA synthesis. The purine salvage pathway is streamlined in Plasmodium, composed of only adenosine deaminase (ADA), purine nucleoside phosphorylase (PNP) and hypoxanthine-guanine-xanthine phosphoribosyltransferase (HGXPRT)¹⁻⁴. Hypoxanthine is a precursor for all purines and a central metabolite for nucleic acid synthesis in P. falciparum⁶. The dual action of PfADA and PfPNP for purines and methylthiopurines permit the parasite to form hypoxanthine from host purine pools and to recycle hypoxanthine from methylthioadenosine, a product of polyamine synthesis. PfADA and PfPNP function at the intersection of the polyamine metabolism⁷⁻⁹ and purine salvage pathway, essential pathways for viability of Plasmodium in its human host.

There remains a pressing need for an effective vaccine against malaria.

### SUMMARY OF THE INVENTION

The inventors have produced an attenuated Plasmodium parasite which confers protection against malaria to a subject. Specifically, the present invention is directed to a Plasmodium parasite having a genome comprising a non-reversible knockout of the PyPNP gene.

The present invention is further directed to a vaccine against malaria comprising a Plasmodium parasite having a genome comprising a non-reversible knockout of the PyPNP gene.

The present invention is further directed to a method for immunizing a subject against malaria comprising administering to the subject the claimed vaccine.

The present invention is further directed to a method for producing a Plasmodium parasite having a genome comprising a non-reversible knockout of the PyPNP gene comprising replacing the PyPNP gene or a portion thereof via double crossover homologous recombination, wherein the double crossover homologous recombination comprises the following steps: (a) transfecting the Plasmodium parasite with a deletion construct comprising a nucleotide sequence identical to the nucleotide sequence located immediately upstream from the PyPNP gene, followed by a drug resistance gene, followed by a nucleotide sequence identical to the nucleotide sequence immediately downstream from the PyPNP gene, (b) contacting the transfected Plasmodium parasite with the drug associated with the drug resistance gene from step (a), and (c) isolating any surviving Plasmodium parasites, wherein any surviving Plasmodium parasite is a Plasmodium parasite having a genome comprising a non-reversible knockout of the PyPNP gene.

The present invention is further directed to a method for producing a vaccine against malaria comprising combining the claimed Plasmodium parasite with a pharmaceutically acceptable carrier.

The present invention is further directed to the use of a Plasmodium parasite for the manufacture of a vaccine and the use of the vaccine for immunizing a subject against malaria.

### BRIEF DESCRIPTION OF THE DRAWINGS

FIGS. 1a-1d. P. yoelii lacking PNP are attenuated. Parasitemia (percentage of infected erythrocytes±standard deviation; panels a & c) and survival curves of mice (panels b & d) from groups of 5 female BALB/c mice (age 8-10 weeks) infected with the indicated parasite lines. Blood smears were made daily, stained with Giemsa, and counted. For experiments with single crossover integrants (Panels a & b), mice were infected with 2×10⁵ infected erythrocytes from WT (wildtype YM; red squares), PyPNP-WT Clone 1 (pnp control integrant), PyPNP-INT Clone 1-3 (pnp disruptant) or PyPNP-INT Clone 2-3 (pnp disruptant). For experiments with double-crossover knock out lines (c & d), mice were infected with 2×10⁴ WT (wildtype YM), ΔPyCSP clone 2-2 (CSP knockout), ΔPyPNP (PNP knockouts made with hDHFR cassette; clones 3-4, 4-2 and 4-5), ΔPyPNP-GFP clone 8 (PNP knockout made with hDHFR-GFP cassette). All mice were infected with parasites on day 0 by tail i.v. injection. For each parasite line, parasitemia was checked daily for 30 days (at least 7 days after parasitemia was no longer detectable).

FIG. 2. Sequence comparison of P. yoelii PNP with Plasmodium and E. coli PNPs. PNP protein sequences from P. yoelii (Py) (SEQ ID NO:1), P. berghei (Pb) (SEQ ID NO:2), P. vivax (Pv) (SEQ ID NO:3), P. knowlesi (Pk) (SEQ ID NO:4), P. falciparum (Pf) (SEQ ID NO:5), P. chabaudi (Pc) (SEQ ID NO:6), and E. coli (Ec) (SEQ ID NO:7) were aligned using ClustalW¹. Amino acids in contact with the transition state analogue ImmH and SO4 at the catalytic site of PfPNP² are shown in green. Asterisks indicate amino acids known to be associated with a hydrophobic pocket in PfPNP that can accommodate the 5′-methylthio group of methylthioinosine (MTI).

FIGS. 3A-3C. Creation of PyPNP-INT and PyPNP-WT parasite clones. Schematic representation of the insertion strategy for gene targeting at the pnp locus of P. yoelii and genotype analysis of PyPNP-INT (A) and PyPNP-WT (B) parasite clones. WT pnp genomic locus and integration generated by either insertion of plasmid pPyPNP-INT or insertion of plasmid pPyPNP-WT via a single cross-over between the homologous pnp sequences. Plasmid pPyPNP-INT or pPyPNP-WT was linearized at the unique BseRI site of the pnp coding sequence. C) Southern blot analysis of HindIII-digested genomic DNA from WT and PyPNP-INT clones (1-3, 1-4, and 2-3) shows that the unique band from WT and two bands in recombinants, when hybridized with pnp probe. Digestion of genomic DNA from WT and PyPNP-WT clones (C1 and C2) with EcoRI, cutting once in pPyPNP-WT but not cutting in pnp homologous sequence, shows that one band from WT and two in recombinants, when hybridized with a probe derived from pnp (nucleotides 32-652 of the pnp ORF).

FIGS. 4A-4D. Creation of PyPNP and PyCSP mutants. Schematic representation of the replacement strategy to generate pnp deletion parasites using a PCR-based gene targeting constructs. The WT pnp genomic locus was targeted by PCR-generated deletion constructs designed to replace the coding region of the pnp gene with either (A) the hdhfr selection cassette (ΔPyPNP) or (B) the hdhfr/gfp selection cassette (ΔPyPNP-GFP) via double cross-over homologous recombination between 5′-fragment and 3′-fragment of the pnp locus that flank the selection cassette. Expected fragments and their predicted size for Southern blot analysis of ΔPyPNP and ΔPyPNP-GFP clones are shown. Primers used for PCR genotyping are indicated as arrows. (C) A similar construct was made for csp using the hdhfr cassette. D) Integration was verified with Southern blot analysis of SpeI-digested genomic DNA from WT and ΔPyPNP clones (3-4, 4-2, and 4-5) and ΔPyPNP-GFP clone 8. Digestion of genomic DNA with SpeI, which cuts once in the dhfr deletion construct but not in the dhfr/gfp deletion construct or in the pnp coding sequence, shows a 4.69 kb band in WT and ΔPyCSP lines (Lanes 1 and 6), a 7.74 kb band from ΔPyPNP-GFP (Lane 5), and a band of 2.08 kb in ΔPyPNP clones (Lanes 2-4) when hybridized with a 5′ flanking region pnp probe.

FIG. 5. Mice immunized with ΔPyPNP are protected from sporozoite challenge from P. yoelii 17XNL. 5 mice immunized with clone C8 of ΔPyPNP and an age matched control group of naive mice were fed upon by 35 mosquitoes infected with P. yoelii 17XNL. Parasitemia was examined by blood smear for 25 days. All mice survived the experiment. 4 immunized mice had detectable infection on days 3-5 (peak parasitemias 0.02%, 0.02%, 0.02%, 0.05% days 3 or 4) that then cleared. Standard deviations of parasitemias are high for control mice because 2 mice had prolonged parasitemias (peak 69% day 15; peak 80% day 16) for 4 days after the other 3 mice (peak parasitemias 29%, 32%, 26% at day 10) had cleared infection.

### DETAILED DESCRIPTION OF THE INVENTION

The present invention provides a Plasmodium parasite having a genome comprising a non-reversible knockout of the PyPNP gene. As used herein, a “knockout” shall refer to any alteration to a gene which renders it inactive. A parasite having a “non-reversible knockout” shall refer to a parasite whose progeny all possess the same knockout (i.e. the progeny to do not revert back to the wild-type genotype). Many genetic techniques are known in the art for rendering a gene inactive, such as deletion of the entire gene or a portion thereof, insertion of one or more nucleotides to the gene, and substitution of one or more nucleotides of the gene. In one embodiment, the knockout comprises a deletion of the entire PyPNP gene. In another embodiment, the knockout comprises a deletion of a portion of the PyPNP gene, an insertion of one or more nucleotides to the PyPNP gene, or a substitution of one or more nucleotides of the PyPNP gene, such that the PyPNP gene is rendered inactive. In the preferred embodiment, the knockout of the PyPNP gene results in an attenuated Plasmodium parasite, wherein the ability of the Plasmodium parasite to cause malaria is nonexistent or reduced.

In the preferred embodiment, the non-reversible knockout of the PyPNP gene is made by replacing the PyPNP gene or a portion thereof via double crossover homologous recombination. For example, the desired portion of the PyPNP gene can be targeted by PCR-generated deletion constructs designed to replace the coding region of the PyPNP gene with other nucleotide sequences. In the preferred embodiment, the coding region of the PyPNP gene is replaced with human dhfr or human dhfr/gfp.

Any of the known species of Plasmodium parasites that can used to produce a Plasmodium parasite having a genome comprising a non-reversible knockout of the PyPNP gene. Examples of Plasmodium parasites include, but are not limited to, Plasmodium falciparum, Plasmodium vivax, Plasmodium ovale, Plasmodium malariae, Plasmodium berghei, Plasmodium knowlesi, Plasmodium chabaudi, and Plasmodium yoelli. In the preferred embodiment, the Plasmodium parasite is Plasmodium falciparum, Plasmodium vivax, Plasmodium ovale, Plasmodium malariae, or Plasmodium knowlesi.

In another embodiment, the genome of the Plasmodium parasite further comprises a knockout of a second gene, resulting in an even greater attenuated Plasmodium parasite. In one embodiment, the second gene is a gene required for viability of the Plasmodium parasite. For example, the second gene can be involved in purine salvage or purine recycling in the Plasmodium parasite. In the preferred embodiment, the second gene encodes adenosine deaminase or hypoxanthine-guanine-xanthine phosphoribosyl-transferase.

The present invention further provides a vaccine against malaria comprising any one of the above claimed Plasmodium parasites and a pharmaceutically acceptable carrier. As used herein, a “pharmaceutically acceptable” carrier shall mean a material that (i) is compatible with the other ingredients of the composition without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include, without limitation, any of the standard pharmaceutical carriers such as phosphate buffered saline solutions, water, emulsions such as oil/water emulsions, microemulsions, and the like.

The present invention further provides a method for immunizing a subject against malaria comprising administering to the subject the above-described vaccine. In the preferred embodiment, the subject is a human.

Numerous modes of vaccine administration are known in the art and can be applied to the claimed vaccine. In the preferred embodiment of the invention, the above-described vaccine can easily be administered parenterally such as for example, by intramuscular, intrathecal, subcutaneous, intraperitoneal, intravenous bolus injection or intravenous infusion. Parenteral administration can be accomplished by incorporating the compounds of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic. Injectable vaccines and vaccine formulations are well known in the art. Examples of such vaccines are described in U.S. Pat. No. 7,371,395, U.S. Pat. No. 7,361,362, and U.S. Pat. No. 7,078,043, all of which are hereby incorporated in their entirety by reference into the subject application.

Additionally, the above-described vaccines can be designed for oral, nasal, lingual, sublingual, nasal, buccal and intrabuccal administration and made without undue experimentation by means well known in the art, for example with an inert diluent or with an edible carrier. The vaccines may be enclosed in gelatin capsules or compressed into tablets. For the purpose of oral therapeutic administration, the vaccines of the present invention may be incorporated with excipients and used in the form of tablets, troches, capsules, elixirs, suspensions, syrups, wafers, chewing gums and the like.

Nasally administered vaccines, which can be delivered via nasal spray or nasal drops, are well known in the art. Examples of such vaccines and methods for administering these vaccines are described in U.S. Pat. No. 7,399,840, U.S. Pat. No. 6,391,318, and U.S. Pat. No. 7,275,537, all of which are hereby incorporated in their entirety by reference into the subject application.

The present invention further provides a method for producing a Plasmodium parasite having a genome comprising a non-reversible knockout of the PyPNP gene comprising replacing the PyPNP gene or a portion thereof via double crossover homologous recombination. In a double crossover or “replacement-type” event, a specific nucleotide sequence on a first DNA strand is replaced by a nucleotide sequence from a second DNA strand. To promote double crossovers, deletion constructs or plasmids are created wherein the replacement nucleotide sequence from the second DNA strand is flanked by nucleotide sequences that correspond to the nucleotide sequences that flank the specific nucleotide to be replaced on the first DNA strand. Preferably, the deletion construct or plasmid comprises a 5′ region, followed by the replacement nucleotide sequence, followed by a 3′ region, wherein at least a portion of the 5′ and 3′ regions of the deletion construct is identical to the 5′ and 3′ regions flanking the nucleotide sequence to be replaced on the first DNA strand. Therefore, upon linearization, the replacement sequence on the second DNA strand is aligned and can cross over with the desired sequence on the first DNA strand. Methods for performing double crossover homologous recombination are well known in the art. Examples of such methods are described in U.S. Pat. No. 7,326,568 and U.S. Pat. No. 7,332,324, the contents of which are hereby incorporated in their entirety by reference into the subject application.

In the preferred embodiment, producing a Plasmodium parasite having a genome comprising a non-reversible knockout of the PyPNP gene comprising replacing the PyPNP gene or a portion thereof via double crossover homologous recombination, wherein the double crossover homologous recombination comprises the following steps: (a) transfecting the Plasmodium parasite with a deletion construct comprising a nucleotide sequence identical to the nucleotide sequence located immediately upstream from the PyPNP gene, followed by a drug resistance gene, followed by a nucleotide sequence identical to the nucleotide sequence immediately downstream from the PyPNP gene, (b) contacting the transfected Plasmodium parasite with the drug associated with the drug resistance gene from step (a), and (c) isolating any surviving Plasmodium parasites, wherein any surviving Plasmodium parasite is a Plasmodium parasite having a genome comprising a non-reversible knockout of the PyPNP gene.

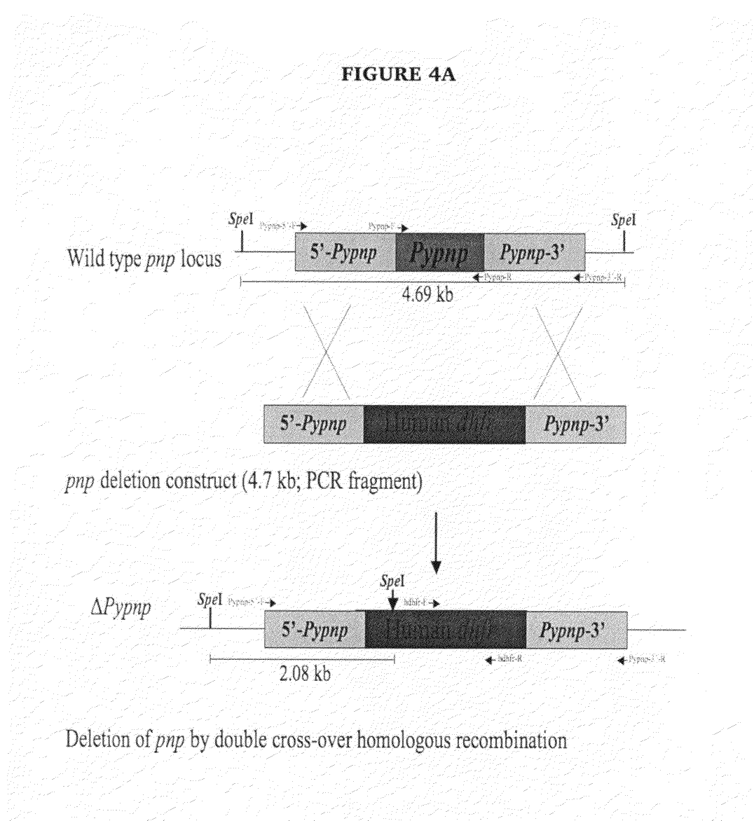

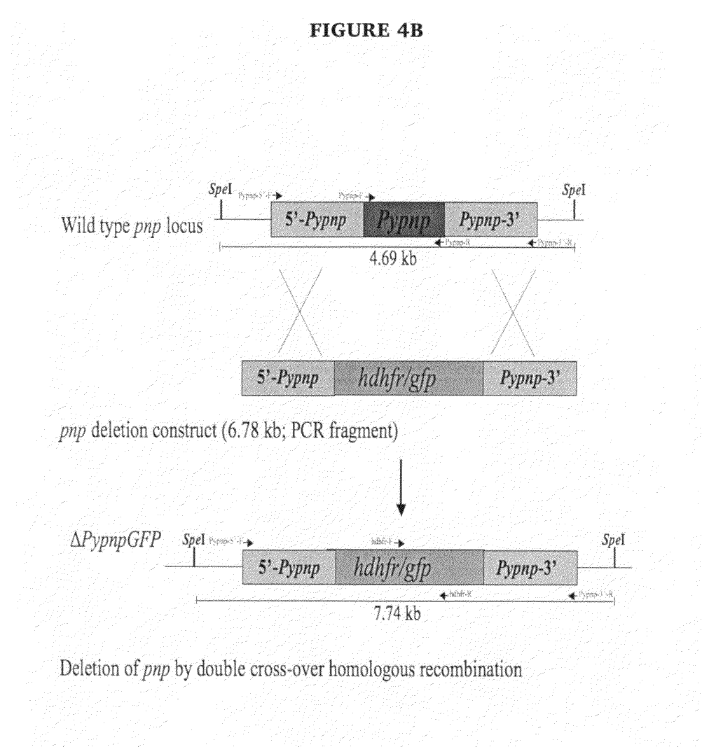

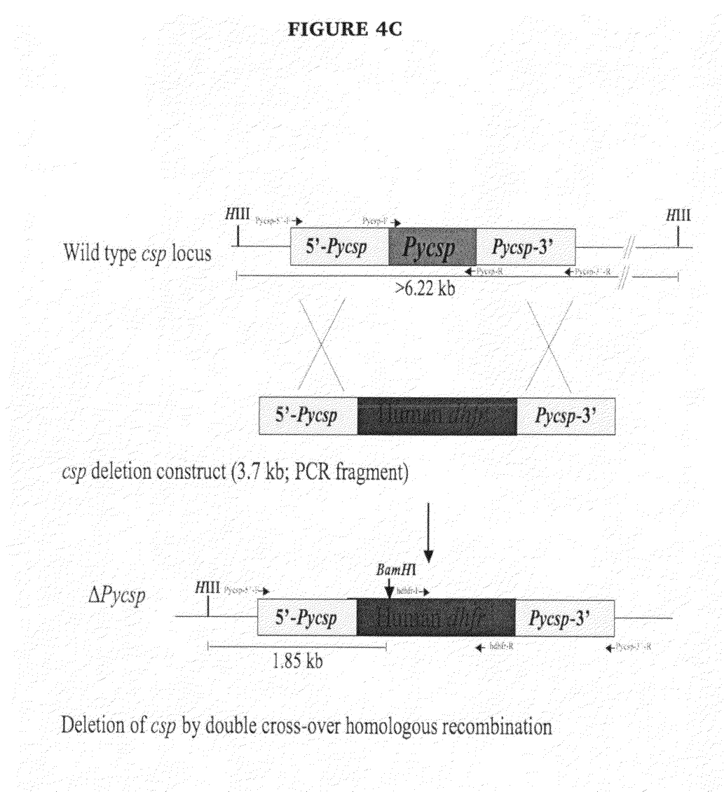

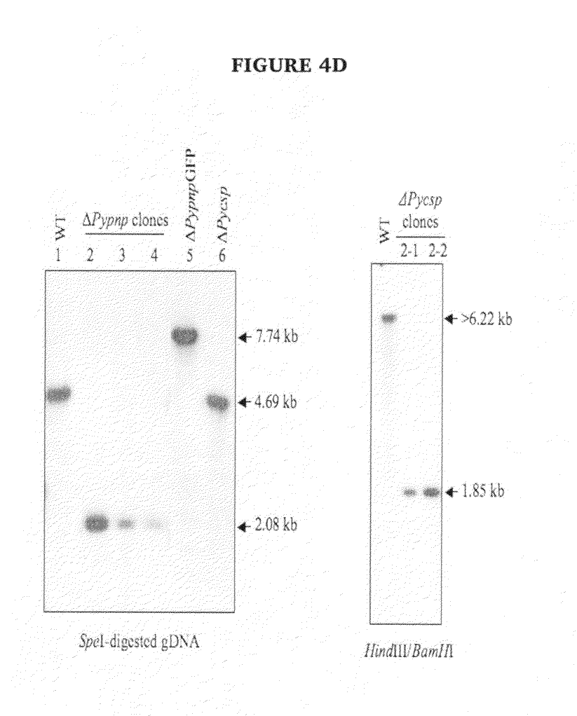

In one embodiment, the portion of the PyPNP gene that is replaced is the coding region of the PyPNP gene. In the claimed method, the coding region of the PyPNP gene can be replaced with any nucleotide sequence that renders the PyPNP gene inactive. Examples of suitable nucleotide sequences include, but are not limited to, sequences which comprise a drug resistance gene, such as human dhfr or human dhfr/gfp. As used herein, a “drug resistance gene” is a gene, which when transfected into a Plasmodium parasite, confers protection to the Plasmodium parasite against the drug associated with the drug resistance gene. As used herein, a “drug associated with the drug resistance gene” shall mean a drug which would otherwise kill a Plasmodium parasite not transfected with the drug resistance gene. In embodiments wherein the PyPNP gene or portion thereof is replaced with human dhfr or human dhfr/gfp, the drug associated with human dhfr or human dhfr/gfp that is administered to the subject is pyrimethamine. Numerous methods for transfecting a Plasmodium parasite are known in the art, which include, but are not limited to, nucleofection, calcium-phosphate mediated transfection, liposome-mediated transfection, sonoporation, heat shock, and magnetofection. In the preferred embodiment, the Plasmodium parasite is transfected with the deletion construct via electroporation. Contacting the transfected Plasmodium parasite with the drug associated with the drug resistance gene can occur in numerous suitable environments. Examples of such suitable environments include in vivo (e.g. in a subject), ex vivo, or in vitro (e.g. on a petri dish) environments. Any suitable non-human mammal can be the subject of the claimed method. Preferably, the transfected Plasmodium parasite is injected into a subject, which is then administered the drug associated with the drug resistance gene. In the preferred embodiment, the subject is a mouse. These preferred embodiments are described below and illustrated in FIG. 4.

The deletion construct used in the above described method can further comprise additional elements. Such elements can be inserted, for example, in between the 5′ nucleotide sequence and the replacement nucleotide sequence, or in between the replacement nucleotide sequence and the 3′ nucleotide sequence. In the preferred embodiment, the additional elements comprise nucleotide sequences which facilitate removal of the deletion construct. However, because the subject invention works best with smaller deletion constructs, it is preferred that the size of the additional elements be kept at a minimum. Site-specific recombination techniques can be used, such that the deletion construct contains specific nucleotide sequences recognizable by an enzyme. Depending on the orientation of these nucleotide sequences with one another within the deletion construct, the enzyme can excise, exchange, integrate or invert the deletion construct. In a preferred embodiment, the drug resistance gene in the deletion construct is flanked on both ends with sequences containing loxP sites. When removal of the drug resistance gene from the Plasmodium parasite genome is desired, the Plasmodium parasite is transfected with Cre recombinase, which cuts the DNA at the loxP sites. Following cutting by Cre recombinase, the Plasmodium parasite DNA, minus the drug resistance gene, can then be rejoined using DNA ligase.

The present invention further provides the Plasmodium parasite having a genome comprising a non-reversible knockout of the PyPNP gene produced by any of the above described methods.

The present invention further provides a method for producing a vaccine against malaria comprising combining the above-claimed Plasmodium parasite with a pharmaceutically acceptable carrier. Examples of suitable pharmaceutically acceptable carriers are described above.

The present invention further provides the use of the above-claimed Plasmodium parasite for the manufacture of a vaccine against malaria and the use of the vaccine for immunizing a subject against malaria.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Materials and Methods

Identification of Plasmodium yoelii PNP (PyPNP). Similarity searches the P. yoelii strain 17XNL genome database with the PfPNP sequence identified a single sequence with significant homology (PyPNP; gene PY04622). The PyPNP gene encodes a single open reading frame corresponding to 244 amino acids protein of molecular mass of 26.943 kDa. The reading frame for PyPNP was amplified by PCR using P. yoelii strain YM genomic DNA as template. DNA sequencing of the PCR product was identical to the data base sequence.

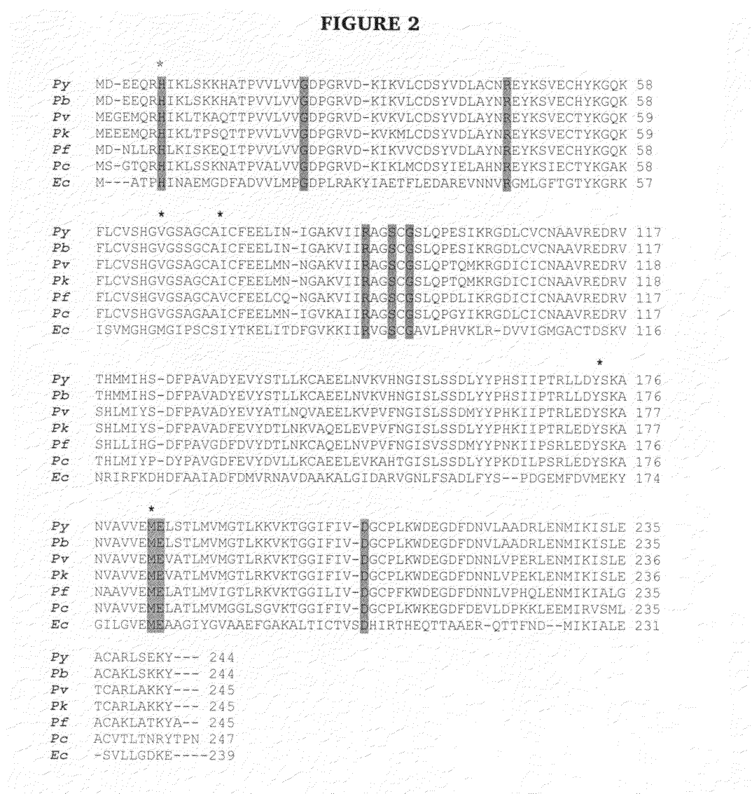

PyPNP exhibits 78% amino acid sequence identity to PfPNP and 23% identity to EcPNP (FIG. 2). All the amino acids in contact with the transition state analogue ImmH and SO₄ in the crystal structure of PfPNP are conserved among these three PNPs. In previous reports, PfPNP was demonstrated to have activity against MTI, a metabolite only present in P. falciparum^{3,4}. The crystal structure of PfPNP with the transition state analogue MT-ImmH shows the methylthio group inserts into a hydrophobic pocket formed by Val-66, Tyr-160, and Met-183 from one subunit and His-7 and Val-73 from a neighboring subunit of the PfPNP hexamer. Sequence alignment shows that four of the five amino acids are conserved with a Val73Ile conservative substitution in PyPNP (FIG. 2). Table 1 shows pairwise comparison showing percentage identity of the nucleotide and amino acid sequences of the PNP gene from six Plasmodium species.

TABLE 1
Pairwise comparison of Plasmodium PNPs

| | |
|---|---|
| Nucleotide | |
| (percentage identity) | Amino acid (percentage identity) |

| Species | Py | Pb | Pv | Pk | Pf | Pc |
|---|---|---|---|---|---|---|
| Py | | 98 | 83 | 82 | 78 | 76 |
| Pb | 93 | | 83 | 82 | 79 | 76 |
| Pv | 71 | 70 | | 95 | 81 | 72 |
| Pk | 74 | 72 | 84 | | 81 | 74 |
| Pf | 78 | 76 | 68 | 69 | | 70 |
| Pc | 78 | 78 | 63 | 67 | 69 | |

Expression, Purification, and Characterization of PyPNP. Recombinant PyPNP was expressed with a C-terminal 6×His tag and the c-myc epitope and purified from induced bacterial culture by nickel affinity chromatography under native conditions. Like PfPNP, recombinant PyPNP elutes from gel filtration chromatography as a single peak consisting of homohexameric protein (≈180 kDa; data not shown). On a denatured SDS-PAGE gel, the protein ran as a single band with a molecular mass of 29.94 kDa. Recombinant PyPNP demonstrated active phosphorylysis activity with various substrates including inosine, MTI, guanosine and deoxyinosine. The catalytic efficiencies (k_{cat}/Kₘ) of PyPNP for these substrates were very similar to those observed in PfPNP (Table 2). When tested with deoxyinosine, a preferred substrate for mammalian PNPs, both PyPNP and PfPNP showed weaker catalytic efficiencies. PyPNP has no detectable activity with uridine (data not shown). In conclusion, PyPNP is enzymatically indistinguishable from its orthologue PfPNP.

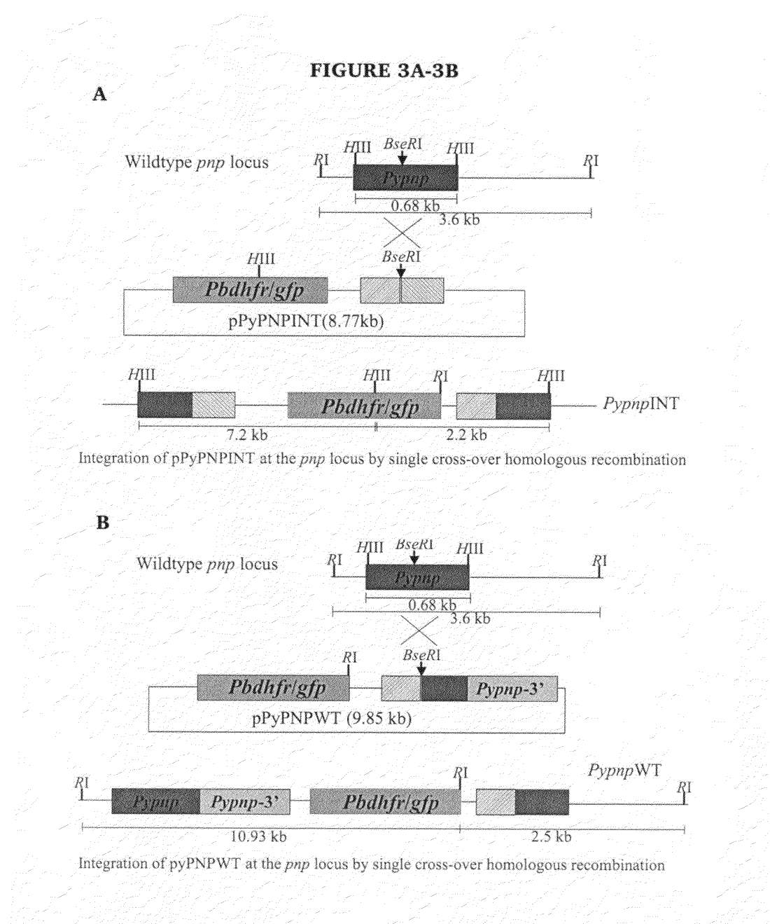

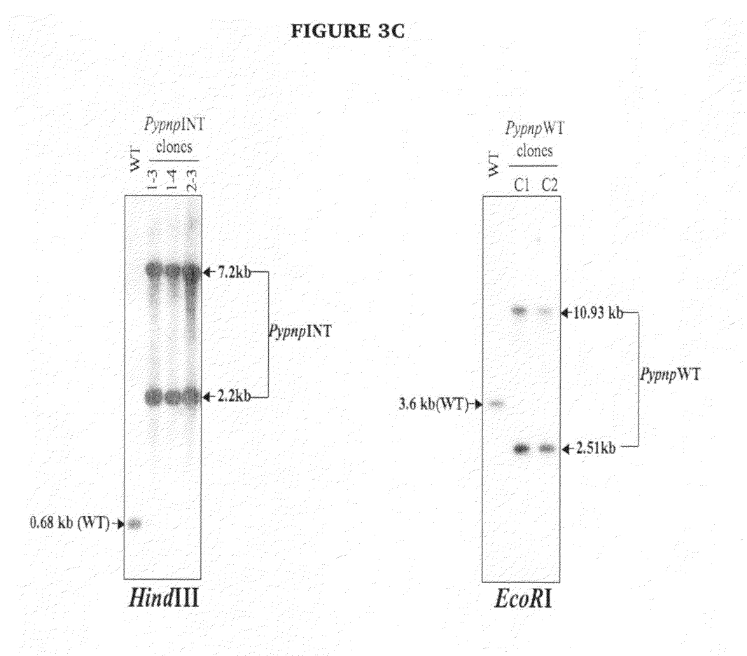

Generation of Transfection Constructs. For gene targeting at pnp genomic locus by insertion, two plasmids were created (FIG. 3). The targeting plasmid pPyPNPINT was constructed by directionally cloning an internal truncated fragment of the PyPNP gene into plasmid pMD205-GFP containing pyrimethamine-resistance Pbdhfr-ts and gfpmut2 fusion gene (Mota 2001) digested with the restriction enzymes BamHI and NotI. The truncated PyPNP fragment was amplified by PCR from P. yoelii strain YM genomic DNA with primers tPyPNP-F (5′-GGCGGATCC CCAAGAAACATGCAACCCCTGT-3′) (SEQ ID NO:8) and tPyPNP-R (5′-GGCGCGGCGC AATGGGATGAGGGAGATTTTG-3′) (SEQ ID NO:9). The underlined sequences represent BamHI and NotI restriction enzyme sites, respectively. The truncated pnp fragment lacks nucleotides 1-31 and the last 83 nucleotides of the pnp ORF. Similarly, knock-in targeting plasmid pPyPNP-WT was constructed by directionally cloning a fragment of 5′-truncated PyPNP gene together with its 3′-downstream region into BamHI-NotI digested pMD205-GFP. The pnp fragment with 3′-fragment was PCR amplified from YM genomic DNA with primers tPyPNP-F (5′-GGCGGATCC CCAAGAAACATGCAACCCCTGT-3′) (SEQ ID NO:8) and PyPNP-WT-R (5′-GGCGCGGCGCTTTTTATTCATGCCCGCTTT-3′) (SEQ ID NO:10). The 5′-truncated pnp fragment with 3′-fragment lacks nucleotides 1-31 of the pnp ORF but contains additional 1026 nucleotides downstream from the stop codon of pnp gene. Before transfection, pPyPNPINT and pPyPNPWT were linearized by BseRI digestion.

For deletion of pnp locus by replacement (FIG. 4), a PCR-based approach for generation of gene targeting constructs for P. berghei was adapted for P. yoelii¹⁷. Two PCR reactions: PCRI and PCRII, required to generate the complete constructs, were carried out by using the Advantage 2 Polymerase Mix (BD Biosciences) and Long Template PCR Polymerase Mix (Roche), respectively, according to the manufacturer's instructions. PCR I amplified two flanking fragments of homology to the pnp gene from P. yoelii genomic DNA using primer combination of PyPNP-5′-F and PyPNP-5′-R or primer combination of PyPNP-5′-F and PyPNP-GFP-5′-R for the 5′-upstream region and primer combination of PyPNP-3′-F and PyPNP-3′-R or primer combination of PyPNP-GFP-3′-F and PyPNP-3′-R for the 3′-downstream region of pnpgene. Primers PyPNP-5′-R and PyPNP-3′-F have 5′-terminal extensions homologous to the selection cassette hdhfr of pL006 (kindly provided by Andy Waters and Chris Janse, Leiden); primers PyPNP-GFP-5′-R and PyPNP-GFP-3′-F have 5′ terminal extensions homologous to the selection cassette hdhfr/gfp of pHDGFP (ref). For PCR II products, the pnp 5′ and 3′ flanking fragments (1.5 kb each) with corresponding extensions from PCR I were combined with either ScalI-linearized pL006 or ScalI-linearized pHDGFP with the outer primers 5′-PyPNP-F and 3′-PyPNP-R. This leads to the amplification of the complete gene targeting constructs for deletion of pnp. Similarly, the circumsporozoite protein (csp) gene deletion construct as an irrelevant gene control was also generated by two-step PCR method that fused 1.0 kb flanking regions of homology with hdhfr selection cassette of pL006.

Primer sequences were as follows:

PyPNP-5′-F (5′-GAATATTTATTCAATGTTTCAAG-3′) (SEQ ID NO:11); PyPNP-5′-R (5′-GCTGGGCTGCAGAGGCCTGTTAACCTACGCAAAAAGGAAGAATATGC-3′) (SEQ ID NO:12); PyPNP-3′-F (5′-CGATGGGTACCCTCGAGGCTAGCGAGACATAAATAAGGAACAAATTCATACATTAC-3′) (SEQ ID NO:13); PyPNP-3′-R (5′-GTATTTTGCTATGGATGTATACAA-3′) (SEQ ID NO:14). Underlined sequences are 5′-terminal overhangs with homology to the hdhfr selection marker of pL006. PyPNP-gfp-5′-R (5′-CATAAAATAATGATTGGGCGAGCTCGCTACGCAAAAAGGAAGAATATGC-3′) (SEQ ID NO:15); PyPNP-gfp-3′-F (5′-GAACATATTTATTAAACTGCAGCCGACATAAATAAGGAACAAATTCATACATTAC-3′) (SEQ ID NO:16). Underlined sequences are 5′-terminal overhangs with homology to the hdhfr/gfp selection marker of pHDGFP. PyCSP-5′-F (5′-AAAATGGGGCTATTTGTACATAAAAG-3′) (SEQ ID NO:17); PyCSP-5′-R (5′-GCTGGGCTGCAGAGGCCTGTTAACTTTAAATATGTGTGTGTATATATAAGTTTTGTTT T-3′) (SEQ ID NO:18); PyCSP-3′-F (5′-CGATGGGTACCCTCGAGGCTAGCGAATAAACATTACACATTATTATAAATATTTA-3′) (SEQ ID NO:19); PyCSP-3′-R (5′-TCAGTTTAGTAACTTTTATATTTATTTTAAATATTT-3′) (SEQ ID NO:20). Underlined sequences are 5′-terminal overhangs with homology to the hdhfr selection marker of pL006.

Transfection and Selection of Stable Transfectants. Blood from P. yoelii wildtype (WT)-infected BALB/c mice was collected by eye vein puncture and set up in culture as previously described¹⁶. Transfection was performed by the AMAXA Nucleofector® (Amaxa GmbH, Germany) using 5-10×10⁷ purified P. yoelii mature schizonts mixed with 100 μl of cytomix (120 mM KCl, 0.15 mM CaCl₂, 10 mM K₂HPO₄/KH₂PO₄, 2 mM EGTA, 5 mM MgCl₂, 25 mM Hepes, pH 7.6) and 10 μg of BseRI-linearized plasmid DNA or PCR products. Parasites were transfected using the electroporation program T-016 available in the Nucleofector® and injected intravenously (tail vein) into naive BALB/c recipient mice as previously described⁴. When transfected with integration constructs, drug resistant parasites PyPNP-INT and PyPNP-WT were selected by oral pyrimethamine treatment with the drinking water of mice (1.75 μg/ml), starting at 24 h post-infection, Mice were treated until parasites were no longer detectable. When the parasitemia reappeared to detectable leils, blood was transferred to new mice via tail i.v. injection to produce parasite numbers sufficient for parasite DNA isolation and analysis. When transfected with deletion constructs, mice were treated with pyrimethamine in drinking water (4.5 μg/ml) continuously until drug-resistant parasites ΔPyPNP, ΔPyPNP-GFP, and ΔPyCSP reappeared. The drug resistant stable transfected parasites were cloned by limiting dilution and injection of single parasites into mice.

Genotypic Analysis of Parasites by Southern Hybridization. For Southern hybridization analysis, equal amount of genomic DNA (2.0 μg) from parasites digested with restriction enzymes, separated on agarose gels, transferred to positively-charged nylon membranes (Amersham Biosciences), hybridized with a DNA probe labeled by random priming with α-[³²P]dATP using a Prime-It Random primer DNA Labeling Kit (Stratagene).

Cloning and Expression of P. yoelii PNP. PyPNP was identified as a protein of 244 amino acids and 26.943 kDa by protein sequence homology to PfPNP (gene PY04622; PlasmoDB.org). The PyPNP open reading frame (ORF) sequence was amplified by PCR from genomic DNA of P. yoelii strain YM with primers PyPNP-F (5′-GATGAGGAACAAAGACATATAAAGC-3′) (SEQ ID NO:8) and PyPNP-R (5′-ATATTTTTCTGATAATCTTGCACAA-3′) (SEQ ID NO:9). The 729 by PCR product was ligated into a pTrcHis2-TOPO vector (Invitrogen) and expressed in E. coli strain TOP 10. Recombinant PyPNP with a C-terminal 6×His tag and the c-myc epitope was induced with 1 mM IPTG at 37° C. for 10 h and purified by nickel chromatography. The purified PyPNP was >95% homogeneous based on denaturing polyacrylamide gel electrophoresis and staining with Coomassie blue. Protein concentration was measured by Bradford protein assay (Bio-Rad).

Enzymatic Assays and Determination of Kinetic Constants. All phosphorylase activity assays were performed with purified enzyme in 50 mM KPO4, pH 7.4 as described¹. Briefly, phosphorylysis of inosine, 2′-deoxyinosine, and 5′-methylthioinosine was measured in a coupled assay with 115 milliunits/ml xanthine oxidase to convert hypoxanthine into uric acid. Uric acid formation was followed by spectrophotometric measurement at 293 nm (E₂₉₃=12.9 mM⁻¹ cm⁻¹). Phosphorylysis of guanosine to guanine was monitored by measuring the disappearance of guanosine at 258 nm (E₂₅₈=5.2 mM⁻¹ cm⁻¹). Adenosine and 5′-methylthioadenosine phosphorylase activities were measured by following the disappearance of the substrate at 265 nm (E₂₅₆=1.9 mM⁻¹ cm⁻¹). Uridine phosphorylase activity was measured by following the conversion of uridine to uracil at 272 nm (E₂₆₀=2.9 mM⁻¹ cm⁻¹). The Michaelis constant Kₘ was determined as the substrate concentration at ½ the maximum velocity. The k_{cat} was determined for each substrate by fits of substrate saturation data to the Michaelis-Menten equation. Values for k_{cat} assume one catalytic site per subunit and are expressed as moles of product per second per mole of subunit.

Phenotypic Analysis of Parasite Growth and Infectivity in Mice. To determine whether the disruption of PyPNP had an effect on parasite growth in vivo, parasite inocula (2×10⁴ or 2×10⁵ blood-stage parasites) were injected intravenously into groups of BALB/c mice (female, 8 weeks of age). A thin blood smear was prepared daily from each infected mouse and stained with Giemsa reagent. For each sample, at least 1000 RBCs were examined by microscopy, and the percent of infected erythrocytes (parasitemia) was calculated. All experiments with mice were conducted in AAALAC approved facilities after review and approval of experimental protocols by the Institutional Animal Care and Use Committee.

Phenotypic Analysis of Parasite Development in Mosquitoes. Anopheles stephensi mosquitoes were infected with P. yoelii parasites, by blood-feeding on two sequential days for 15 min on infected Swiss Webster mice and subsequently maintained under 80% humidity at 24° C. The presence of gametocytes was monitored on Giemsa-stained blood smears before mosquito blood meal. Infected mosquitoes were dissected at days 8 and 15 after the first infectious blood meal to evaluate the development of parasites by counting the numbers of oocysts per midgut and the numbers of sporozoites per salivary gland. The number of sporozoites per salivary gland was determined by mixing the salivary glands of 20 infected mosquitoes in 200 μl of PBS and counting the numbers of sporozoites in duplicate in a hemocytometer. Statistical significance was analyzed by ANOVA using the GraphPad Prism software package.

### Results and Discussion

In this study, genetic approaches were used to investigate the importance of Plasmodium PNP in vivo in the rodent malaria P. yoelii (lethal strain YM). P. yoelii is a favored model for vaccine development because of its high infectivity to laboratory mice, particularly in sporozoite infection of hepatocytes¹⁰⁻¹⁴. P. yoelii YM, like P. falciparum, infects both reticulocytes and mature erythrocytes, whereas nonlethal P. yoelii 17XNL and another rodent malaria, Plasmodium berghei, infect only reticulocytes. Because rodent and human erythrocytes have ample PNP and purine salvage activity, erythrocytes may provide sufficient purines to sustain Plasmodium parasites in infected animals.

The PNP genes from all Plasmodium species are highly conserved with PfPNP being 78% identical at the amino acid level to PyPNP (FIG. 2). Detailed biochemical analysis showed that recombinant PyPNP has substrate specificity and kinetic properties similar to PfPNP (Table 2).

TABLE 2
Kinetic constants of Plasmodium PNP

| | P. yoelii PNP | | | P. falciparum PNP | | |
|---|---|---|---|---|---|---|
| | Kₘ | k_{cat} | k_{cat}/Kₘ | Kₘ | k_{cat} | k_{cat}/Kₘ |
| Substrate | (μM) | (s⁻¹) | (M⁻¹ s⁻¹) | (μM) | (s⁻¹) | (M⁻¹ s⁻¹) |
| Inosine | 8.6 ± 1.5 | 3.5 ± 0.1 | 4.0 × 10⁵ | 4.7 ± 0.9 | 1.1 ± 0.2 | 2.3 × 10⁵ |
| Methylthioinosine | 5.0 ± 0.4 | 3.4 ± 0.1 | 6.8 × 10⁵ | 10.8 ± 0.9 | 2.6 ± 0.8 | 2.4 × 10⁵ |
| Guanosine | 8.9 ± 1.6 | 3.8 ± 0.2 | 4.2 × 10⁵ | 9.4 ± 1.2 | 2.6 ± 0.5 | 2.8 × 10⁵ |
| 2′-deoxyinosine | 79 ± 10 | 1.2 ± 0.1 | 1.5 × 10⁴ | 91 ± 35 | 0.9 ± 0.3 | 9.8 × 10³ |

The PyPNP gene was disrupted using a single-crossover insertion strategy^{15,16} (FIG. 3). The viability of PNP-disruptant P. yoelii clones (PyPNP-INT) was compared to a recombinant parasite control with insertion into the PNP-encoding locus without disruption (PyPNP-WT). When PyPNP-INT parasite clones were passaged in mice without drug selection, wild-type revertants developed and rapidly overgrew the PyPNP-INT population (data not shown), providing initial genetic evidence that parasites lacking PyPNP were less fit than wild-type parasites. To prevent reversion, a PCR approach¹⁷ was adopted to generate PyPNP knock-out clones with the genes encoding the selectable markers human dihydrofolte reductase (hDHFR, ΔPyPCP clones) or hDHFR-gfp, a fusion protein of hDHFR and green fluorescent protein¹⁸ (ΔPyPNP-GFP clones) replacing PyPNP by double cross-over homologous recombination (FIG. 4). In parallel, a control strain lacking the gene encoding the circumsporozoite protein or CSP (ΔPyCSP), a gene that is not essential in erythrocytic stages¹⁹, was created.

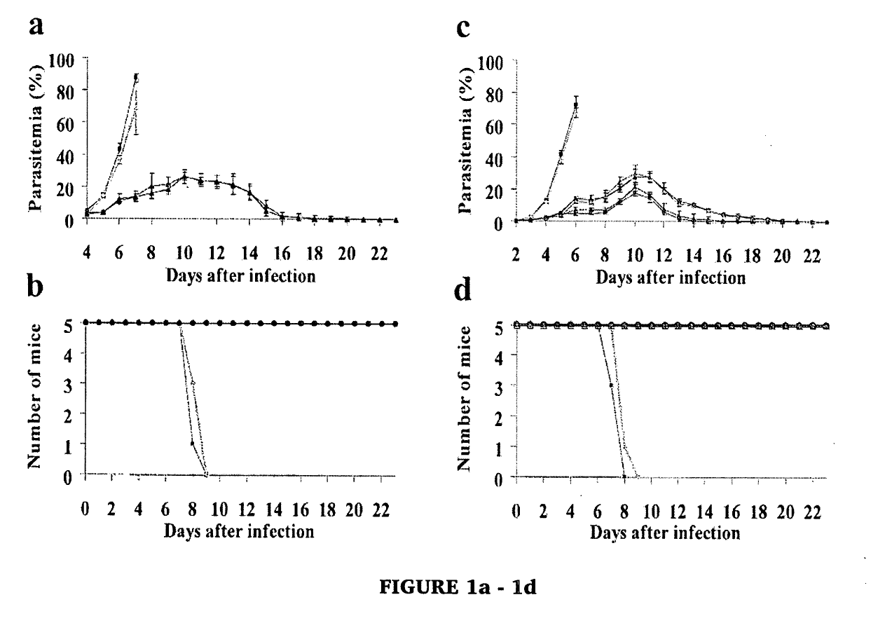

The virulence of PNP-deficient clones was compared to recombinant controls by infecting BALB/c mice and monitoring parasitemia for 30 days (FIG. 1). The growth rate of PyPNP-INT parasites was slower than that of the wild-type P. yoelii YM strain and of PyPNP-WT (FIG. 1a). All WT-infected and PyPNP-INT-infected mice developed high parasitemias and died within ten days of challenge (FIG. 1a, b). Similarly, ΔPyPNP and ΔPyPNP-GFP parasites had no detectable impairment of intraerythrocytic development, but growth of these lines was impaired compared to that of WT or ΔPyCSP parasites (FIG. 1c, d). None of the parasite lines lacking PyPNP (PyPNP-INT, ΔPyPNP and ΔPyPNP-GFP) were lethal to mice, and infected mice recovered from infection with no detectable parasitemia (FIG. 1b, d). The PyPNP-INT, ΔPyPNP and ΔPyPNP-GFP clones were also attenuated in Swiss Webster mice (data not shown). Thus PNP is critical for optimal replication of malaria parasites during the erythrocytic cycle, despite the significant levels of PNP activity present in mammalian erythrocytes.

There are other points in the life cycle where parasites have significant purine requirements. The sexual-stage gametocytes are taken up by mosquitoes from mammalian blood. In the mosquito midgut, male gametocytes undergo exflagellation, a process that involves four rapid nuclear divisions and results in formation of eight microgametes within fifteen minutes. Ookinetes develop from the fertilized zygote and migrate across the mosquito midgut where they form oocysts. Thousands of sporozoites are made within each oocyst. These sporozoites invade mosquito salivary glands and are injected into the mammalian host. After sporozoite invasion of hepatocytes, the parasites develop into exoerythrocytic forms that undergo extensive multiplication, eventually releasing tens of thousands of merozoites into the circulation.

Oocyst and sporozoite development in Anopheles stephensi mosquitoes fed on mice infected with WT, PyPNP-WT, PyPNP-INT or ΔPyPNP parasite clones bearing similar levels of mature gametocytes were analyzed. At day eight post-feeding, PyPNP-INT-infected mosquitoes had markedly fewer numbers of oocysts compared to WT- or PyPNP-WT-infected mosquitoes, and no ΔPyPNP oocysts were detected (Table 3). On day fifteen post-feeding, PyPNP-INT- or ΔPyPNP-infected mosquitoes had no detectable sporozoites. The numbers of ΔPyCSP oocysts were normal, but infected mosquitoes had no sporozoites in their salivary glands, confirming that CSP is essential for development of P. yoelii sporozoites in mosquitoes, as previously described for P. berghei¹⁹. Thus PNP activity is important for blood stage development and critical for mosquito stage development.

TABLE 3
Parasite development in mosquitoes

| | | No. of | No. of salivary | No. of infected/No. | |
|---|---|---|---|---|---|
| | | midgut oocysts | gland sporozoites | of mice bitten | Pre-patent period |
| Experiment | Parasite clone | per mosquito^{a} | per mosquito | by mosquitoes | (days)^{c}/genotype^{d} |
| 1 | WT clone | 47.7 ± 10.2 | 11,875 | 2/2 | d 3/WT |
| | PyPNP-WT C1 | 45.4 ± 6.5 | 11,000 | 2/2 | d 3/PyPNP-WT |
| | PyPNP-INT | 2.8 ± 0.6 | 0 | 0/2 | —/— |
| | C1-3 | | | | |
| 2 | WT clone | 42.8 ± 7.5 | 7,750 | 2/2 | d 3/WT |
| | PyPNP-WT C2 | 40.6 ± 7.4 | 8,000 | 2/2 | d 3/PyPNP-WT |
| | PyPNP-INT | 1.5 ± 0.5 | 0 | 1/2 | d 4/WT^{e} |
| | C1-4 | | | | |
| 3 | WT clone | 46.8 ± 7.9 | 8,875 | 2/2 | d 3/WT |
| | ΔPyCSP C2-2 | 41.4 ± 10.1 | 0 | 0/2 | —/— |
| | ΔPyPNP C4-5 | 0 | 0 | 0/2 | —/— |
| | ΔPyPNP C8 | 0 | 0 | 0/2 | —/— |

^{a}Mean number of oocysts per midgut was determined by dissecting at least 20 mosquitoes at day 8. All 3 experiments show statistically significant differences by ANOVA p < 0.0001
^{b}Mean number of sporozoites per salivary gland was determined by dissecting 20 mosquitoes at day 15.
^{c}Number of days after mosquito bite until detectable blood stage parasites by microscopic examination of blood smear.
^{d}Genotype of blood stage parasites recovered after sporozoite infection by mosquitoes was analyzed by PCR using genomic DNA as template. PyPNP-WT is the control recombinant whereas WT indicates the original wild-type PNP genotype.
^{e}WT parasites were recovered from a mouse bitten by mosquitoes infected with PyPNP-INT, suggesting that WT revertants are able to develop into infectious sporozoites. Similar reversion was described during characterization of P. berghei TRAP single crossover disruptants³⁰.

Because attenuated microorganisms are frequently used as vaccines, the ability of PNP-knockout parasites to confer protection against subsequent malaria infection was tested (Table 4). Naive mice challenged with PyPNP-INT, ΔPyPNP or ΔPyPNP-GFP survived and remained parasite-free. These mice were rechallenged six to eight weeks later with a lethal dose of WT P. yoelii YM parasites (2×10⁵ infected erythrocytes). Immunized mice were completely protected with none developing detectable parasitemia (Table 4).

TABLE 4
Protection of immunized mice against challenge with P. yoelii WT

| | | Immunization | | |
|---|---|---|---|---|
| | | with parasite | No. protected/ | |
| Exper- | Mouse | clone/No. of | challenged | Detection of |
| iment^{a} | Strain | parasites | (% protected) | Parasitemia^{b} |
| 1 | BALB/c | PyPNP-INT C1-4/ | 10/10 (100) | Not detectable |
| | | 2.0 × 10⁵ | | |
| 2 | BALB/c | PyPNP-INT C1-3/ | 5/5 (100) | Not detectable |
| | | 2.0 × 10⁵ | | |
| | BALB/c | PyPNP-INT C2-3/ | 5/5 (100) | Not detectable |
| | | 2.0 × 10⁵ | | |
| 3 | BALB/c | None | 0/5 (0) | Day 2 |
| | BALB/c | ΔPyPNP^{c}/ | 20/20 (100) | Not detectable |
| | | 2.0 × 10⁴ | | |
| 4 | BALB/c | None | 0/5 (0) | Day 2 |
| | Swiss | PyPNP-INT C1-4/ | 3/3 (100) | Not detectable |
| | Webster | 1.0 × 10⁷ | | |
| 5 | Swiss | PyPNP-INT C1-3/ | 5/5 (100) | Not detectable |
| | Webster | 1.0 × 10⁷ | | |
| | Swiss | None | 0/5 (0) | Day 2 |
| | Webster | | | |
| 6 | Swiss | ΔPyPNP C4-5/ | 3/3 (100) | Not detectable |
| | Webster | 1.0 × 10⁷ | | |
| | Swiss | ΔPyPNP C8/ | 3/3 (100) | Not detectable |
| | Webster | 1.0 × 10⁷ | | |
| | Swiss | None | 0/3 (0) | Day 2 |
| | Webster | | | |

^{a}Age matched mice were used as naive controls. All mice were challenged with an intravenous inoculum of 2.0 × 10⁵ P. yoelii YM infected erythrocytes.
^{b}Blood smears were monitored daily for 14 days. Number of days indicates when parasitemia was detectable after mice were challenged with WT P. yoelii YM.
^{c}A total 20 female BALB/c mice divided into groups of 5 were infected with ΔPyPNP clones C3-4, C4-2, C4-5, or ΔPyPNP-GFP C8 (5 mice per clone). These mice were later rechallenged with 2.0 × 10⁵ P. yoelii 17XNL at 7 months age and did not develop detectable parasitemia.

The protection conferred by infection with the attenuated PNP-deficient parasites was long-lasting and also provided protection to the nonlethal 17XNL strain of P. yoelii. Twenty ΔPyPNP-immunized mice surviving a lethal challenge with YM (Experiment 3; Table 4) had no detectable parasitemia after challenge with P. yoelii 17XNL erythrocytic stages five months after initial exposure to ΔPyPNP. Age-matched control mice (5 mice) developed parasitemias up to 50% and then cleared the infection (data not shown).

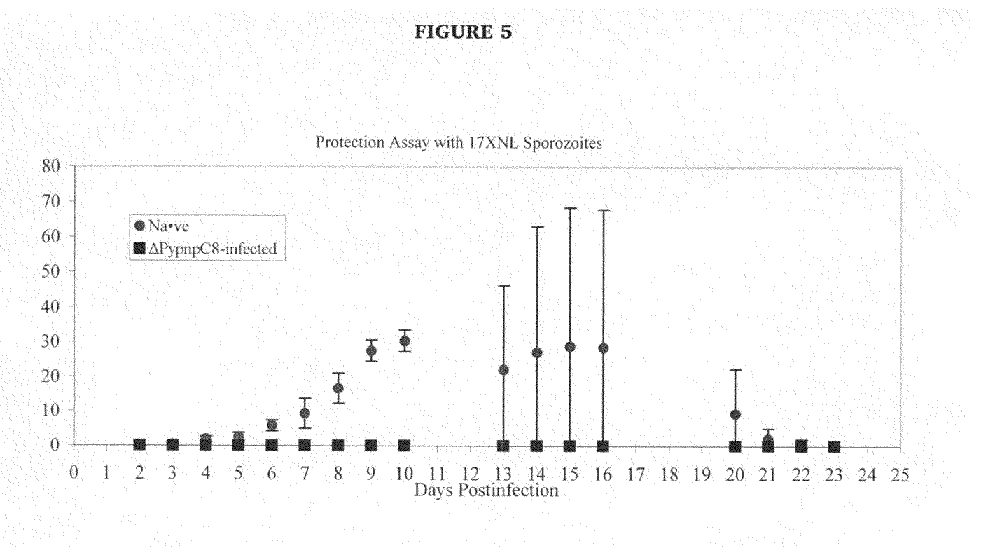

Mice were also protected from 17XNL sporozoite challenge (FIG. 5). Five BALB/c mice inoculated with 2.0×10⁴ P. yoelii YM ΔPyPNP were exposed to 35 A. stephensi mosquitoes infected with P. yoelii 17XNL. Four of these mice developed low levels of parasitemia on days 3-5 that resolved within 3 days with maximal peak parasitemia of 0.05%. The naive mice all developed high parasitemias (up to 80%) but then cleared the infection (FIG. 5).

Attenuated whole-parasite vaccines, particularly irradiated attenuated sporozoites, have been proposed as alternatives to subunit vaccines²⁰⁻²². Whereas the implementation of such vaccines poses considerable logistical difficulties, better protection has been elicited with attenuated sporozoites than with candidate subunit vaccines²². More recently, genetically attenuated sporozoites have been developed²³. Whole-parasite vaccines may circumvent the problems resulting from heterogeneous host responses to individual antigens by providing an array of conserved immunogenic proteins less subject to immune pressure than current subunit vaccine components²⁴.

A small study in humans has suggested that low levels of malaria infection may be protective in humans²⁵. This study was performed by drug-curing infected individuals and suggests that repeated low-level malaria infection may be protective²⁵. Routine administration of chemotherapy is constrained by the wide-spread resistance to commonly used antimalarials and has the potential to enhance the rate of selection of drug-resistant organisms. An alternative approach might be vaccination with a highly attenuated asexual stage parasite that clears spontaneously.

Development of a live attenuated vaccine strain requires a balance between the level of attenuation and the immunogenicity of the vaccine to optimize safety and efficacy. Some level of parasite replication will probably be required to mount an effective immune response^{26,27}. The parasitemias detected after infection with PNP-deficient parasites are higher than would likely be tolerated in humans, but the studies illustrate that development of attenuated erythrocytic stage metabolic mutants is feasible. Although attenuated sporozoite vaccines are promising, their success is dependent upon complete protection, as a single successful sporozoite infection with development of merozoites within hepatocytes could lead to symptomatic malaria²⁸. In contrast, even a partially effective erythrocytic stage malaria vaccine could have significant clinical impact on morbidity and mortality by decreasing the incidence of severe disease such cerebral malaria.

Ideally, genetically engineered Plasmodium vaccine strains will not revert to wild-type virulence but be able to establish a self-limited, subclinical and protective course of infection. Since disruption of P. falciparum PfNT1²⁹, a purine nucleoside transporter, results in parasites that cannot survive at physiological concentrations of purines, parasite strains with multiple purine-salvage deletions will probably be more highly attenuated than single-deletion strains. Parasite strains lacking PNP still make gametocytes and may have activity as transmission blocking vaccines while not being transmissible. While non-transmissible vaccines as favored due to their safety, it is possible that attenuated vaccine strains that can infect mosquitoes will promote protective immunity beyond the vaccine and therefore be more beneficial to the general population. Finally, these studies establish the importance of Plasmodium purine salvage enzymes for in vivo survival and support continued exploration of purine salvage as a chemotherapeutic target.

### REFERENCES

1. Kicska, G. A. et al. Transition state analogue inhibitors of purine nucleoside phosphorylase from Plasmodium falciparum. J Biol Chem 277, 3219-25 (2002).
2. Kicska, G. A. et al. Purine-less death in Plasmodium falciparum induced by immucillin-H, a transition state analogue of purine nucleoside phosphorylase. J Biol Chem 277, 3226-31 (2002).
3. Shi, W. et al. Plasmodium falciparum purine nucleoside phosphorylase: crystal structures, immucillin inhibitors, and dual catalytic function. J Biol Chem 279, 18103-6 (2004).
4. Ting, L. M. et al. Targeting a novel Plasmodium falciparum purine recycling pathway with specific immucillins. J Biol Chem 280, 9547-54 (2005).
5. Snow, R. et al. Estimating mortality, morbidity and disability due to malaria among Africa's non-pregnant population. Bull. World Health Organ. 77, 624-640 (1999).
6. Reyes, P. et al. Enzymes of purine and pyrimidine metabolism from the human malaria parasite, Plasmodium falciparum. Mol Biochem Parasitol 5, 275-90 (1982).
7. Muller, S. et al. In the human malaria parasite Plasmodium falciparum, polyamines are synthesized by a bifunctional ornithine decarboxylase, S-adenosylmethionine decarboxylase. J Biol Chem 275, 8097-102 (2000).
8. Muller, S., Coombs, G. H. & Walter, R. D. Targeting polyamines of parasitic protozoa in chemotherapy. Trends Parasitol 17, 242-9 (2001).
9. Wrenger, C., Luersen, K., Krause, T., Muller, S. & Walter, R. D. The Plasmodium falciparum bifunctional ornithine decarboxylase, S-adenosyl-L-methionine decarboxylase, enables a well balanced polyamine synthesis without domain-domain interaction. J Biol Chem 276, 29651-6 (2001).
10. Burkot, T. R., Graves, P. M., Paru, R., Wirtz, R. A. & Heywood, P. F. Human malaria transmission studies in the Anopheles punctulatus complex in Papua New Guinea: sporozoite rates, inoculation rates, and sporozoite densities. Am J Trop Med Hyg 39, 135-44 (1988).
11. Weiss, W. R., Good, M. F., Hollingdale, M. R., Miller, L. H. & Berzofsky, J. A. Genetic control of immunity to Plasmodium yoelii sporozoites. J Immunol 143, 4263-6 (1989).
12. Weiss, W. R. Host-parasite interactions and immunity to irradiated sporozoites. Immunol Lett 25, 39-42 (1990).
13. Khan, Z. M. & Vanderberg, J. P. Role of host cellular response in differential susceptibility of nonimmunized BALB/c mice to Plasmodium berghei and Plasmodium yoelii sporozoites. Infect Immun 59, 2529-34 (1991).
14. Hoffman, S. L. et al. Immune response gene regulation of immunity to Plasmodium berghei sporozoites and circumsporozoite protein vaccines. Overcoming genetic restriction with whole organism and subunit vaccines. J Immunol 142, 3581-4 (1989).
15. Mota, M. M., Thathy, V., Nussenzweig, R. S. & Nussenzweig, V. Gene targeting in the rodent malaria parasite Plasmodium yoelii. Mol Biochem Parasitol 113, 271-8 (2001).
16. Jongco, A. M., Ting, L. M., Thathy, V., Mota, M. M. & Kim, K. Improved transfection and new selectable markers for the rodent malaria parasite Plasmodium yoelii. Mol Biochem Parasitol 146, 242-50 (2006).
17. Ecker, A., Moon, R., Sinden, R. E. & Billker, O. Generation of gene targeting constructs for Plasmodium berghei by a PCR-based method amenable to high throughput applications. Mol Biochem Parasitol 145, 265-8 (2006).
18. Kadekoppala, M. et al. Stable expression of a new chimeric fluorescent reporter in the human malaria parasite Plasmodium falciparum. Infect. Immun. 68, 2328-2332 (2000).
19. Menard, R. et al. Circumsporozoite protein is required for development of malaria sporozoites in mosquitoes. Nature 385, 336-40 (1997).
20. Wykes, M. & Good, M. F. A case for whole-parasite malaria vaccines. Int J Parasitol 37, 705-12 (2007).
21. Hoffman, S. L. et al. Protection of humans against malaria by immunization with radiation-attenuated Plasmodium falciparum sporozoites. J Infect Dis 185, 1155-64 (2002).
22. Luke, T. C. & Hoffman, S. L. Rationale and plans for developing a non-replicating, metabolically active, radiation-attenuated Plasmodium falciparum sporozoite vaccine. J Exp Biol 206, 3803-8 (2003).
23. Mueller, A. K., Labaied, M., Kappe, S. H. & Matuschewski, K. Genetically modified Plasmodium parasites as a protective experimental malaria vaccine. Nature 433, 164-7 (2005).
24. Makobongo, M. O. et al. The purine salvage enzyme hypoxanthine guanine xanthine phosphoribosyl transferase is a major target antigen for cell-mediated immunity to malaria. Proc Natl Acad Sci USA 100, 2628-33 (2003).
25. Pombo, D. J. et al. Immunity to malaria after administration of ultra-low doses of red cells infected with Plasmodium falciparum. Lancet 360, 610-7 (2002).
26. Sambandamurthy, V. K. & Jacobs, W. R., Jr. Live attenuated mutants of Mycobacterium tuberculosis as candidate vaccines against tuberculosis. Microbes Infect 7, 955-61 (2005).
27. Scheller, L. F. & Azad, A. F. Maintenance of protective immunity against malaria by persistent hepatic parasites derived from irradiated sporozoites. Proc Natl Acad Sci USA 92, 4066-8 (1995).
28. Bejon, P. et al. Calculation of liver-to-blood inocula, parasite growth rates, and preerythrocytic vaccine efficacy, from serial quantitative polymerase chain reaction studies of volunteers challenged with malaria sporozoites. J Infect Dis 191, 619-26 (2005).
29. El Bissati, K. et al. The plasma membrane permease PfNT1 is essential for purine salvage in the human malaria parasite Plasmodium falciparum. Proc Natl Acad Sci USA 103, 9286-91 (2006).
30. Sultan, A. A. et al. TRAP is necessary for gliding motility and infectivity of Plasmodium sporozoites. Cell 90, 511-522 (1997).

What is claimed is:

1. A Plasmodium parasite having a genome comprising a non-reversible knockout of the PyPNP gene.

2. The Plasmodium parasite of claim 1, wherein the non-reversible knockout of the PyPNP gene is made by replacing the PyPNP gene or a portion thereof via double crossover homologous recombination.

3. The Plasmodium parasite of claim 2, wherein the portion of the PyPNP gene is the coding region of the PyPNP gene.

4. The Plasmodium parasite of claim 1, wherein the Plasmodium parasite is Plasmodium yoelii.

5. The Plasmodium parasite of claim 1, wherein the knockout comprises a deletion of the entire PyPNP gene.

6. The Plasmodium parasite of claim 1, wherein the knockout comprises a deletion of a portion of the PyPNP gene, an insertion of one or more nucleotides to the PyPNP gene, or a substitution of one or more nucleotides of the PyPNP gene, such that the PyPNP gene is rendered inactive.

7. The Plasmodium parasite of claim 1, wherein the genome of the Plasmodium parasite further comprises a knockout of a second gene.

8. The Plasmodium parasite of claim 7, wherein the second gene is a gene required for viability of the Plasmodium parasite.

9. The Plasmodium parasite of claim 8, wherein the second gene is a gene involved in purine salvage or purine recycling.

10. The Plasmodium parasite of claim 9, wherein the second gene encodes adenosine deaminase or hypoxanthine-guanine-xanthine phosphoribosyl-transferase.

11. A vaccine against malaria comprising the Plasmodium parasite of claim 1 and a pharmaceutically acceptable carrier.

12. A method for immunizing a subject against malaria comprising administering to the subject the vaccine of claim 11.

13. The method of claim 12, wherein the subject is a human.

14. The method of claim 12, wherein the vaccine is administered via intramuscular, intrathecal, subcutaneous, intraperitoneal, intravenous bolus injection or intravenous infusion.

15. A method for producing a Plasmodium parasite having a genome comprising a non-reversible knockout of the PyPNP gene comprising replacing the PyPNP gene or a portion thereof via double crossover homologous recombination, wherein the double crossover homologous recombination comprises the following steps:
(a) transfecting the Plasmodium parasite with a deletion construct comprising a nucleotide sequence identical to the nucleotide sequence located immediately upstream from the PyPNP gene, followed by a drug resistance gene, followed by a nucleotide sequence identical to the nucleotide sequence immediately downstream from the PyPNP gene;
(b) contacting the transfected Plasmodium parasite with the drug associated with the drug resistance gene from step (a); and
(c) isolating any surviving Plasmodium parasites,
wherein any surviving Plasmodium parasite is a Plasmodium parasite having a genome comprising a non-reversible knockout of the PyPNP gene.

16. The method of claim 15, wherein portion of the PyPNP gene is the coding region of the PyPNP gene.

17. The method of claim 15, wherein the Plasmodium parasite is transfected with the deletion construct via electroporation.

18. The method of claim 15, wherein the drug resistance gene is human dhfr or human dhfr/gfp.

19. The method of claim 18, wherein the drug is pyrimethamine.

20. A Plasmodium parasite having a genome comprising a non-reversible knockout of the PyPNP gene produced by the method of claim 15.

21. A method for producing a vaccine against malaria comprising combining the Plasmodium parasite of claim 1 with a pharmaceutically acceptable carrier.
